(19) United States
(12) Patent Application Publication
(10) Pub. No.: US 2014/0142458 A1
(43) Pub. Date: May 22, 2014

(54) IMPLANTABLE MONITORING DEVICE WITH SELECTABLE REFERENCE CHANNEL AND OPTIMIZED ELECTRODE PLACEMENT

(71) Applicant: CYBERONICS, INC., Houston, TX (US)

(72) Inventors: Kent W. Leyde, Sammamish, WA (US); Jaideep Mavoori, Mercer Island, WA (US)

(21) Appl. No.: 14/163,117

(22) Filed: Jan. 24, 2014

Related U.S. Application Data
(63) Continuation-in-part of application No. 13/441,609, filed on Apr. 6, 2012.
(60) Provisional application No. 61/473,639, filed on Apr. 8, 2011.
(60) Provisional application No. 61/756,319, filed on Jan. 24, 2013.
(60) Provisional application No. 61/756,330, filed on Jan. 24, 2013.

(51) Int. Cl. H03F 3/68; A61B 5/0476; H03F 1/34

(52) U.S. Cl. CPC H03F 3/68; H03F 1/34; A61B 5/0476
USPC 600/544; 330/124 R; 330/84

(57) ABSTRACT

Techniques for amplifying a plurality of input voltages to generate a corresponding plurality of output voltages. In an exemplary embodiment, each of the plurality of input voltages is referenced to a common voltage comprising the average of the plurality of input voltages, wherein for each of the input voltages, the common voltage is coupled to a common node via a corresponding switch.

### CROSS REFERENCE TO RELATED APPLICATIONS

The present application is a continuation-in-part of U.S. patent application Ser. No. 13/441,609, filed Apr. 6, 2012, which claims the benefit of U.S. Provisional Application No. 61/473,639, filed Apr. 8, 2011; and further claims the benefit of U.S. Provisional Patent Application No. 61/756,330, filed Jan. 24, 2013, and U.S. Provisional Patent Application No. 61/756,319, filed Jan. 24, 2013, the complete disclosures of which are incorporated by reference herein in their entirety.

### TECHNICAL FIELD

The present disclosure relates generally to techniques for designing sensing devices that are implantable in a body of a patient.

### BACKGROUND

Implantable biomedical devices may utilize component microelectronic circuitry implanted in the body of a patient to perform functions benefiting the health of the patient. For example, in the field of neurological monitoring, multiple electrodes may be implanted in diverse locations near, on, or in a patient's brain to monitor cortical potentials (Electro Encephalogram, or EEG). This data may be subsequently processed in order to determine if a patient is experiencing a seizure, or is at elevated susceptibility to experiencing a seizure. See, e.g., U.S. patent application Ser. No. 12/020,450, “Systems and Methods for Identifying a Contra-ictal Condition in a Subject,” filed Jan. 25, 2008, assigned to the assignee of the present application, the contents of which are hereby incorporated by reference in their entirety.

The design of signal conditioning front-end circuitry for implantable biomedical devices calls for robust and accurate signal sensing capabilities that minimize the effects of external environmental signal sources and the effects of unrelated physiological processes, while effecting minimal disturbance to the patient. A number of different amplification approaches may be used to measure neurological potentials. An often used approach involves measuring the difference in potential between two adjacent electrodes. Because the electrodes are nearby each other, they tend to be affected in the same way by interfering sources such as external static potentials, as interfering signals tend to manifest themselves as common to both channels (“common-mode”). By measuring only the difference in potential between the two electrodes (“differential-mode”), common-mode interference signals may be rejected.

A well-designed system will address factors that prevent common-mode signals from being converted into differential-mode signals (said to be an artifact of the interfering signal), which may be quantified by the “Common Mode Rejection Ratio”, or CMRR. Achieving a high CMRR typically requires the use of circuit components with tight tolerances and circuits that embody symmetrical features. While differential measurement approaches tend to provide good rejection of certain types of interfering signals, they also introduce measurement issues. In particular, the ability to resolve the location where a signal is being generated becomes an issue. This is because, in a differential system, it may be unclear as to which of the two electrodes being used in the measurement is sensing the signal.

Other measurement approaches may be used that attempt to measure the signal associated with a single sensing electrode. This is often the case when using implanted sub-dural monitoring electrodes. Use of the signal from a single electrode may help to better localize a region of interest, such as brain tissue that is associated with seizure initiation, i.e., a “seizure onset zone”. The ability to measure the potential associated with a single electrode may also have advantages for use with algorithms such as seizure advisory algorithms. In practice, potentials cannot be measured alone, they must be measured in comparison with another potential. To measure the signal from a single electrode requires the designation of a reference point or reference electrode. A fundamental limitation is that unwanted signal appearing on the reference electrode cannot be distinguished from signal arising on the sensing electrode. For this reason, the reference electrode should be chosen or designed to be as free from signal as possible.

In conventional neuro-amplification systems, a designated reference channel is provided. A user will attempt to place the associated reference electrode in an area that is electrically “quiet,” meaning that the location is largely free from neuro-potentials, myographic potentials, and interfering environmental signals. By its very nature, the reference electrode location tends to be well separated from the area where the desired neuro-potentials are being measured. A typical reference location choice would be the vertex of the patient's head. This location is relatively distant from underlying muscle and associated artifact and tends to exhibit smaller neuro-potential signals. The separation of the reference electrode and the sensing electrodes means that interfering sources may act on the electrodes differently, arising in artifact that is difficult or impossible to remove. When used as part of an implantable system, the separation of the measuring electrodes from the reference electrode could also lead to a need for a more complex system, the need for additional surgical incisions, and increased risk of complications.

### SUMMARY

It would be desirable to create a neuro-amplification system that is able to provide a reasonably quiet reference potential so that signals from single electrodes can be measured in relative isolation. It would also be desirable to minimize the number of electrodes required for the system. This could be accomplished by utilizing all of the electrodes for sensing the signal of interest. Furthermore, it would be desirable to avoid the need for placing a reference electrode at a distant location from other electrodes positioned near the source of the signal of interest.

It would be further desirable to minimize artifact caused by myographic potentials, or by environmental static potentials. It would also be desirable to minimize electrical potentials generated in the body of a patient due to operation of the component circuitry, as well as any residual current leaking from the device into the patient. Furthermore, it would be desirable to provide techniques for automatically detecting mechanical and/or electrical failure of the sensing device, so that appropriate actions may be taken to address such failure.

In accordance with embodiments of the present invention, an apparatus for amplifying a plurality N of inputs to generate N outputs is provided. The apparatus comprises: a first stage amplifier for amplifying each of the N inputs relative to a first common reference to generate N intermediate outputs, the first common reference comprising the average of the N inputs; and a second stage amplifier for amplifying each of the N intermediate outputs relative to a second common reference to generate the N outputs, the second stage amplifier comprising the average of the N intermediate outputs.

In accordance with embodiments of the present invention, an apparatus for amplifying a plurality N of inputs to generate N outputs is provided, the apparatus comprising: an amplifier for amplifying each of the N inputs relative to a common reference to generate the N outputs, the common reference comprising the average of the N inputs; and a memory coupled to the N outputs, the memory configured to record each of the N outputs.

In accordance with embodiments of the present invention, an apparatus is provided, comprising: a plurality N of electrical input leads; and an amplifier for amplifying voltages at each of the N electrical input leads relative to a common reference to generate N outputs, the common reference comprising the average of the N inputs; wherein each of the N electrical input leads is coupled to a corresponding physiological signal source to be measured.

In accordance with embodiments of the present invention, an apparatus is provided, comprising: a plurality N of electrical input leads; and an amplifier for amplifying voltages at each of the N electrical input leads relative to a common reference to generate N outputs, the common reference comprising the average of the N inputs; wherein none of the N electrical input leads is coupled to a designated reference electrode.

In accordance with embodiments of the present invention, a method is provided comprising: coupling a plurality N of inputs to a physiological signal source; and amplifying the N inputs relative to a common reference to generate N outputs, the common reference comprising the average of the N inputs.

In accordance with embodiments of the present invention, an apparatus for amplifying a plurality N of inputs to generate N outputs is provided, the apparatus comprising: an amplifier for amplifying each of the N inputs relative to a common reference to generate the N outputs, the common reference comprising the average of the N inputs; and a signal processing module configured to process the plurality of output voltages, the signal processing module comprising: a summation module configured to sum the plurality of output voltages; an out-of-range detection module configured to detect when the output of the summation module exceeds a pre-defined range.

In accordance with embodiments of the present invention, a method is provided, comprising: amplifying a plurality N of input voltages using a first stage to generate N first voltages, the amplifying comprising referencing each of the N input voltages to a first common voltage reference, the first common voltage reference comprising the average of the plurality N of input voltages; and amplifying the N first voltages using a second stage to generate N output voltages, the amplifying the N first voltages comprising referencing each of the N first voltages to a second common voltage reference, the second common voltage reference comprising the average of the N first voltages.

In accordance with embodiments of the present invention, an apparatus is provided, comprising: a plurality N of input conducting leads; and amplifier means to amplify the voltages at each of the plurality N of input conducting leads referenced to a common voltage, the common voltage comprising the average of the voltages at the plurality N of input conducting leads.

In accordance with embodiments of the present invention, an apparatus is provided, comprising: a housing; a plurality of input conducting leads; and a plurality of amplifier modules contained in the housing, each amplifier module comprising an input node coupled to a corresponding one of the plurality of input conducting leads, each amplifier module further comprising at least one corresponding output node; wherein a bias node conductively couples a bias voltage to the input node of each amplifier module.

### BRIEF DESCRIPTION OF THE DRAWINGS

FIG. 1 illustrates an exemplary embodiment of an implantable apparatus according to the present disclosure.

FIG. 2 illustrates an exemplary embodiment of the plurality of amplifier modules.

FIG. 3 illustrates an exemplary embodiment of the apparatus wherein input protection circuitry is shown.

FIG. 4 illustrates an alternative exemplary embodiment wherein a clamp is provided in the bias arrangement for additional voltage protection.

FIG. 5 illustrates an alternative exemplary embodiment of the apparatus having impedance measurement capability at the amplifier module inputs.

FIG. 6 illustrates an exemplary embodiment of a calibration voltage generation module.

FIG. 7 illustrates an exemplary embodiment of a method for measuring the impedance between input nodes IN of two amplifier modules using the apparatus of FIG. 5.

FIG. 8A illustrates an exemplary embodiment of a signal processing module configured to generate an anomaly indicator signal.

FIG. 8B illustrates an exemplary embodiment of the present disclosure, wherein insulating sheaths are provided for portions of the conducting leads external to the housing.

FIG. 9 illustrates a monitoring system having implanted electrodes in communication with an external assembly through an implanted monitoring device.

FIG. 10 depicts an exemplary embodiment of the present disclosure, wherein the techniques disclosed hereinabove are applied in the context of a real-time patient monitoring and neurological event detection system.

FIG. 11 illustrates exemplary configurations of electrode arrays.

FIG. 12 illustrates a monitoring assembly that may be implanted beneath one or more layers of the scalp.

FIG. 13 illustrates a lead assembly.

FIG. 14 illustrates a plurality of amplifier modules in accordance with another embodiment.

FIG. 15 illustrates a plurality of amplifier modules in accordance with another embodiment.

FIG. 16 illustrates an electrode placement over the temporalis muscle.

FIG. 17 illustrates another electrode placement over the temporalis muscle.

### DETAILED DESCRIPTION

The detailed description set forth below in connection with the appended drawings is intended as a description of exemplary embodiments of the present invention and is not intended to represent the only exemplary embodiments in which the present invention can be practiced. The term “exemplary” used throughout this description means “serving as an example, instance, or illustration,” and should not necessarily be construed as preferred or advantageous over other exemplary embodiments. The detailed description includes specific details for the purpose of providing a thorough understanding of the exemplary embodiments of the invention. It will be apparent to those skilled in the art that the exemplary embodiments of the invention may be practiced without these specific details. In some instances, well known structures and devices are shown in block diagram form in order to avoid obscuring the novelty of the exemplary embodiments presented herein.

In this specification and in the claims, it will be understood that when an element is referred to as being “connected to” or “coupled to” another element, it can be directly connected or coupled to the other element or intervening elements may be present. In contrast, when an element is referred to as being “directly connected to” or “directly coupled to” another element, there are no intervening elements present. When two elements are referred to as being “conductively coupled” to one another, then the two elements are coupled by a path having non-zero conductance, or finite resistance. For example, there may be a short-circuit path (i.e., a path of very high conductance) between two “conductively coupled” elements, or there may be a resistive path between such two “conductively coupled” elements.

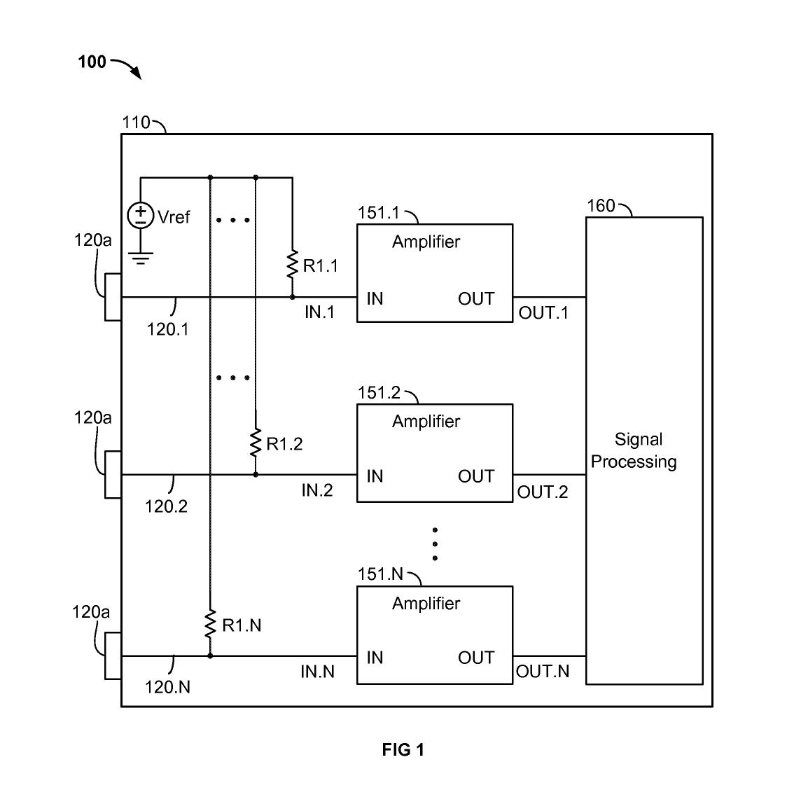

FIG. 1 illustrates an exemplary embodiment of an implantable apparatus 100. Note the apparatus 100 is shown for illustrative purposes only, and is not meant to limit the scope of the present disclosure. In alternative exemplary embodiments, an apparatus may incorporate any or all of the features shown in FIG. 1, and such alternative exemplary embodiments are contemplated to be within the scope of the present disclosure.

In FIG. 1, the apparatus 100 includes a housing or case 110. In an exemplary embodiment, the housing may be made of titanium, ceramic, or other types of biocompatible material, e.g., non-conductive biocompatible materials. The housing is coupled to a plurality of input conducting leads or terminals 120.1 through 120.N. Note N denotes herein the number of channels processed by the apparatus, and n will denote an integer index from 1 to N. Each conducting lead 120.n is designed to sense electrical potentials present on such tissue or fluid. Each conducting lead 120.n may come in direct physical contact with, e.g., biological tissue or fluid inside the body of a patient, or be coupled to another lead which carries the detected signal. Each of the conducting leads 120.n is coupled to a corresponding input node IN.n of a plurality of amplifier modules 151.1 through 151.N.

The input to each amplifier module 151.n may be biased by a reference voltage Vref through a corresponding resistor R1.n, also denoted a bias resistance. The amplifier modules 151.1 through 151.N collectively amplify input voltages at IN.1 through IN.N to generate output voltages at OUT.1 through OUT.N, which are provided to a signal processing module 160. The module 160 may perform further signal conditioning on the amplifier module outputs, as well as analog-to-digital conversion for further processing by a digital computational module (not shown).

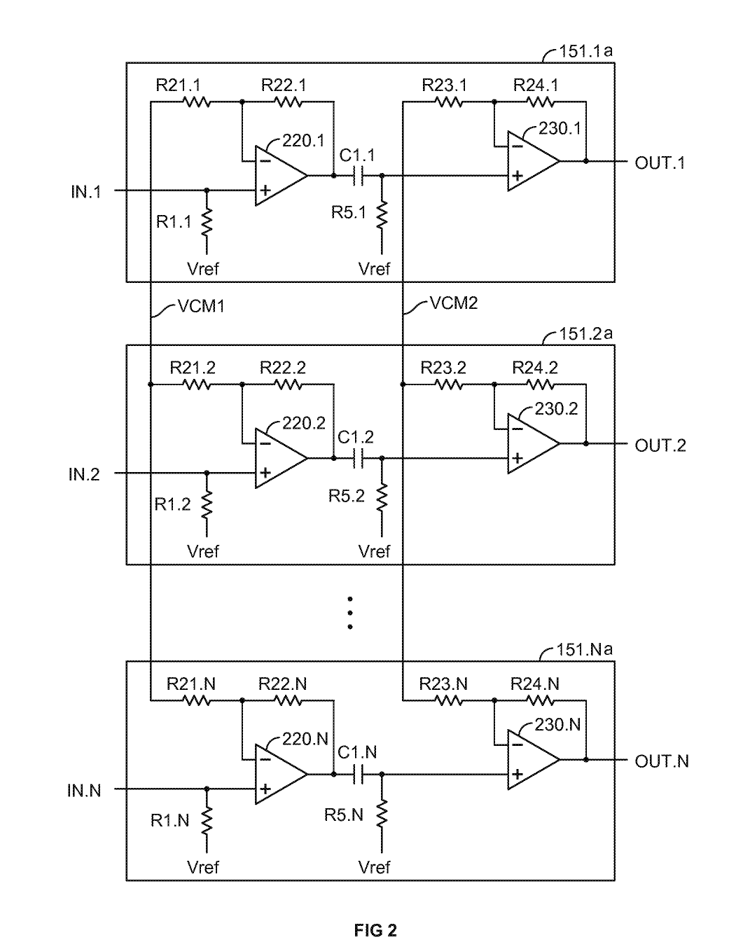

Note the amplifier modules 151.1 through 151.N shown in FIG. 1 may, in some embodiments, be inter-connected with each other using electrical couplings not shown, as later described herein with reference to FIG. 2. The illustration of FIG. 1 is not meant to limit the possible types of inter-connections between amplifier modules.

The apparatus 100 may amplify and process the plurality of input voltages IN.1 through IN.N without necessarily referencing a common voltage, e.g., a ground voltage.

FIG. 2 illustrates an exemplary embodiment of amplifier modules 151.1 through 151.N. Note for ease of illustration, FIG. 2 omits certain details that will be clear to one of ordinary skill in the art, e.g., power supply voltages, additional provision of filtering networks in the circuit, addition or omission of components in the feedback networks of each op amp, etc. Furthermore, FIG. 2 is not intended to limit the implementation of the amplifier modules 151.1 through 151.N in FIG. 1 to that shown in FIG. 2, and one of ordinary skill in the art will appreciate that certain aspects of the present disclosure may readily be applied to alternative implementations of the amplifier modules 151.1 through 151.N.

In FIG. 2, each amplifier module 151.n of FIG. 1 is implemented as a corresponding amplifier module 151.na. For example, amplifier module 151.1a includes a first differential amplifier 220.1 and a second differential amplifier 230.1. In an exemplary embodiment, each of the differential amplifiers 220.1 and 230.1 may be, e.g., an operational amplifier known in the art. In an exemplary embodiment, the non-inverting inputs to each differential amplifier 220.1 and 230.1 may be biased to Vref via resistors R1.1 and R5.1, respectively. It will be appreciated that by biasing all patient-connected conductive leads, e.g., conductive leads 120.1 through 120.N, to a single reference voltage Vref, leakage and/or corrosion in the circuit may be advantageously minimized.

In an exemplary embodiment, the bias voltage Vref may be chosen to be at approximately halfway between supply voltages used to power the amplifiers 220 and 230, thereby maximizing the available signal swing at the non-inverting input nodes to both op amps.

In FIG. 2, the non-inverting input of amplifier 220.1 is coupled to the input voltage node IN.1 of amplifier module 151.1. The output of amplifier 220.1 is fed back to its inverting input via the resistive division of resistors R21.1 and R22.1. In alternative exemplary embodiments, it will be appreciated that the resistances R21.1 and R22.1 may be implemented as generalized impedances, and may be denoted herein as the first inverting input impedance and the first feedback impedance, respectively. The output of amplifier 220.1 is further coupled to a coupling capacitor C1.1, which is in turn coupled to the non-inverting input of amplifier 230.1. The output of amplifier 230.1 is similarly fed back to its inverting input via the resistive division of resistors R23.1 and R24.1. In alternative exemplary embodiments, it will be appreciated that the resistances R23.1 and R24.1 may also be implemented as generalized impedances, and may be denoted herein as the second inverting input impedance and the second feedback impedance, respectively. Furthermore, the coupling capacitor C1.1 may also be replaced by a generalized coupling impedance, and may be implemented using passive or active elements. Such alternative exemplary embodiments are contemplated to be within the scope of the present disclosure.

In FIG. 2, each of the amplifier modules 151.1a through 151.Na may be implemented as described hereinabove with reference to amplifier module 151.1a. For example, each amplifier module 151.na may include resistors R21.n and R22.n for feedback of the output of the first differential amplifier 220.n to its inverting input. Note resistors R21.1 through R21.N across all amplifiers 220.1 through 220.N are coupled together at a single common node VCM1. Similarly, resistors R23.1 through R23.N across all amplifiers 230.1 through 230.N are coupled together at a single common node VCM2.

It will be appreciated that the topology shown in FIG. 2 configures each amplifier module 151.n to generate a corresponding output voltage at OUT.n by amplifying the difference between the corresponding input voltage at IN.n and the average of all the input voltages at IN.1 through IN.N. The topology thus advantageously provides for the amplification of N input signals to generate N output signals for further processing, advantageously without the need to additionally reference an externally provided common reference voltage, e.g., a ground voltage. This distinguishes the architecture from other instrumentation amplifiers found in the prior art, wherein one of the N input signals would generally need to be coupled to a “quiet” or otherwise physically separate reference voltage. Furthermore, prior art instrumentation amplifiers are generally not capable of providing N output voltages for N input voltages, unless a separate voltage, e.g., a ground voltage, is also referenced at the input and/or the output.

By eliminating the need to provide a physically separate reference voltage, as earlier described in the Background section, the amplifier architecture of FIG. 2 further advantageously simplifies the design of the sensing system, and further eliminates the potential need for additional surgical incisions, and increased risk of complications. Furthermore, it will be appreciated that the architecture of FIG. 2 provides improved common-mode rejection, compared to prior art amplifiers that require a separate reference voltage.

Note the configuration of amplifiers 220.1 through 220.N, along with feedback networks, may be referred to as a composite “first-stage amplifier” in the present disclosure, and in the claims. Similarly, amplifiers 230.1 through 230.N, along with feedback networks, may be referred to as a composite “second-stage amplifier.”

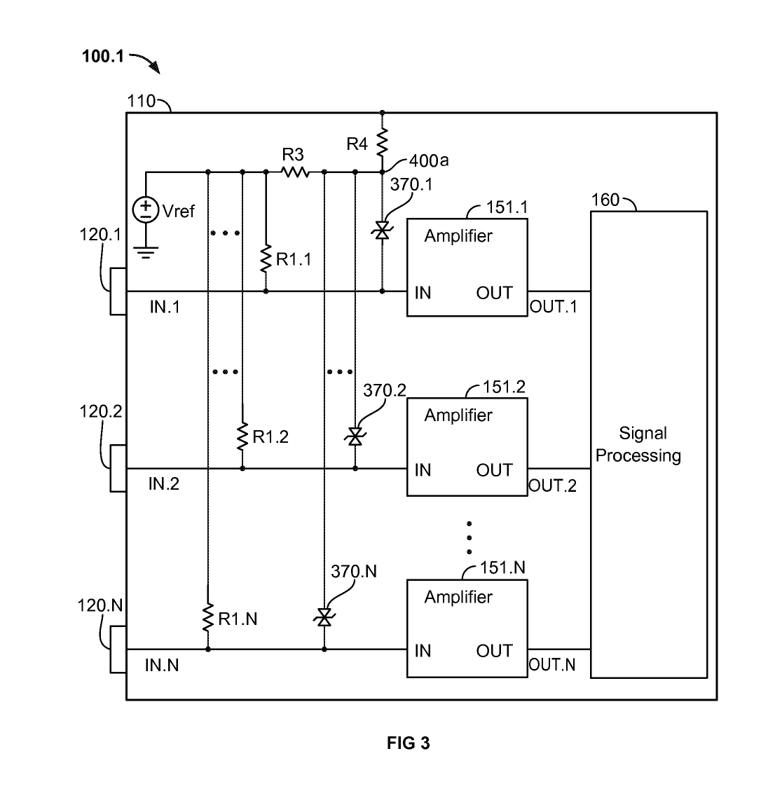

In an aspect of the present disclosure, input protection circuitry is further provided to the apparatus 100 to protect against possible adverse electrical events. FIG. 3 illustrates an exemplary embodiment 100.1 of the apparatus 100 wherein input protection circuitry is shown. In FIG. 3, one end of a bidirectional Zener diode 370.1 is coupled to the input node IN.1 of amplifier 151.1, while another end of the diode 370.1 is coupled to a node 400a. Similarly, diodes 370.2 through 370.N are provided for amplifier modules 151.2 through 151.N. Note all diodes 370.1 through 370.N share the common node 400a.

It will be appreciated that the diodes 370.1 through 370.N may function to prevent excessive voltage from being built up between any of inputs IN.1 through IN.N. Note the connection of node 400a to Vref through resistor R3 keeps corresponding input nodes IN.1 through IN.N biased within the range of the input protection devices 410, and eliminates the voltage across diodes 370.1 through 370.N and hence leakage through them, thereby also reducing unwanted noise generation. One of ordinary skill in the art will appreciate that in alternative exemplary embodiments, other devices may be used in place of the bidirectional Zener diodes shown, e.g., unidirectional Zener diodes, other clamping devices known in the art, etc.

Further shown in FIG. 3 is a resistor R4 coupling node 400a to the housing 110. It will be appreciated that such a configuration may advantageously minimize potential differences amongst the internal bias of amplifier modules 151.1 through 151.N, the housing 110, and the conducting leads 120.1 through 120.N. In an exemplary embodiment, all components (including the housing 110) that are in contact with the patient may be coupled to the same reference voltage. In an exemplary embodiment, R4 may have a suitably high resistance, e.g., 1 Gigaohm, to maximize resistance from Vref to the housing, thereby minimizing current flow and maintaining high common-mode rejection ratio (CMRR). Further note the provision of R4 may advantageously prevent static build-up when the apparatus is disposed outside of the human body, and also limit leakage current between, e.g., the housing and ground.

Note in alternative exemplary embodiments, other resistive networks (not shown) may be provided in place of, or in addition to, R3 and R4 shown in FIG. 3 to accomplish similar functions to those described. For example, parallel paths and/or multiple resistors may be provided between any of the housing 110, Vref, and the nodes IN.1 through IN.N, to offer more conductive paths. Such alternative exemplary embodiments are contemplated to be within the scope of the present disclosure.

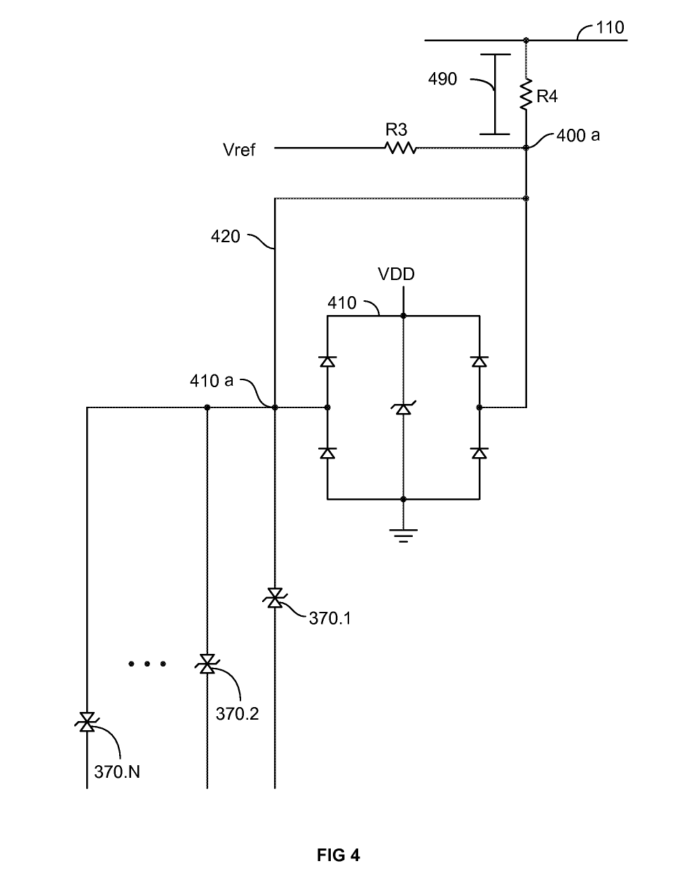

FIG. 4 illustrates an alternative exemplary embodiment wherein a clamp 410 is provided in the bias arrangement for additional voltage protection. In FIG. 4, the outputs of the diodes 370.1 through 370.N are jointly coupled to a node 410a, while a clamp 410 clamps nodes 410a and 400a to a range between VDD and GND. A short circuit path 420 is also provided in parallel with the clamp 410. It will be appreciated that the clamp 410 provides multiple electrical paths wherein current at the input nodes IN.1 through IN.N may be shunted to the supply voltage VDD or to ground through the diodes depicted in the clamp 410.

In an exemplary embodiment, for further protection from large voltages accumulating between the housing 110 and any other circuit element, an air gap may be provided between the housing 110 and any reference voltage or any circuit element protected against static discharge, e.g. any of the Zener diodes shown in FIG. 4. In FIG. 4, an exemplary air gap 490 is shown between the housing 110 and the node 400a. It will be appreciated that other air gaps not shown in FIG. 4 may also be provided in the device according to the principles described herein, and such alternative exemplary embodiments are contemplated to be within the scope of the present disclosure.

The air gap 490 may be implemented by limiting the physical separation between the housing 110 and the node 400a to be less than a maximum distance, e.g., 1 millimeter. The air gap 490 thus effectively acts as a parallel path to the resistance R4 to discharge any large voltage potentials between the housing 110 and the node 400a through electrical arcing resulting from breakdown of a gas medium, e.g., helium, between the housing 110 and the node 400a.

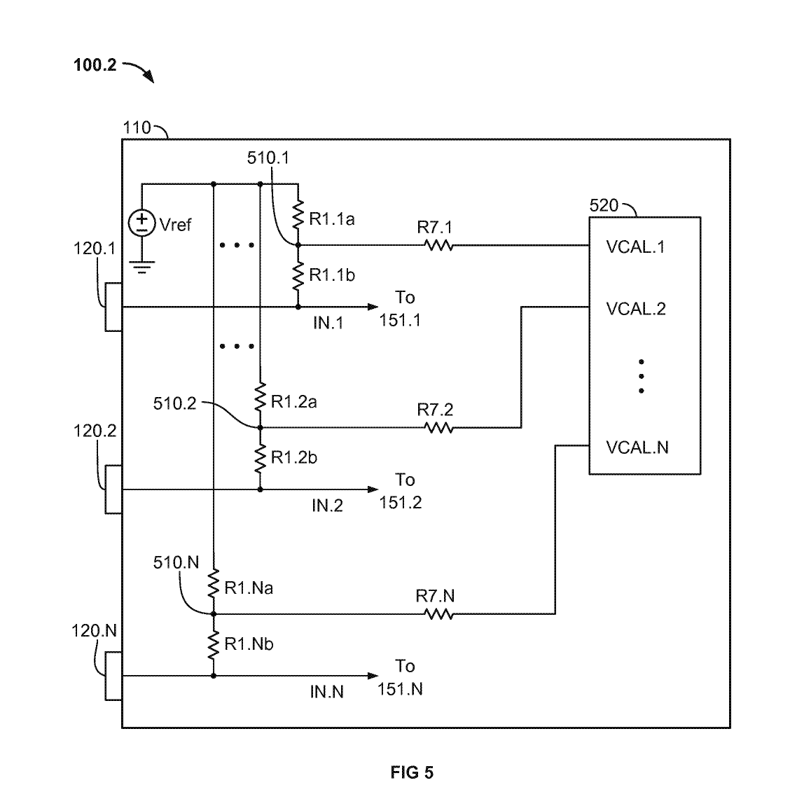

In a further aspect of the present disclosure, input impedance measurement capability is provided for the apparatus 100. FIG. 5 illustrates an alternative exemplary embodiment 100.2 of the apparatus 100 having impedance measurement capability at the amplifier module inputs. In FIG. 5, the resistance R1.n biasing the input IN.n to each amplifier module is split into two component series resistances R1.na and R1.nb. For example, the resistance R1.1 coupling the reference voltage Vref to IN.1 is tapped at an internal node 510.1, thereby splitting the resistance R1.1 into two resistances R1.1a and R1.1b. Each internal node 510.n, also denoted the calibration node, may be further coupled via a resistance R7.n to a corresponding calibration voltage VCAL.n generated by a calibration module 520. In an exemplary embodiment, the module 520 may generate VCAL.n using a programmable voltage source, e.g., as an output of a digital-to-analog converter on a microprocessor (not shown), or as selected from amongst a plurality of input voltages to a multiplexer. In another embodiment, the module 520 generates VCAL.n using standard digital outputs, e.g., from a microprocessor switching between the microprocessor's low and high digital voltages, utilizing the resistances R7.n and R1.na to attenuate the microprocessor's output voltages VCAL.n to the desired level. By utilizing the microprocessor's standard low and high digital voltages to generate VCAL.n, it is possible to avoid the need to manage additional digital-to-analog converters. Each calibration voltage VCAL.n may include, e.g., DC (static) or AC (time-varying) waveforms.

In an exemplary embodiment, measurement of the impedance between any two input nodes IN.x and IN.y, wherein x and y are each an integer index from 1 to N, and x y, may proceed as described hereinbelow. In an illustrative case wherein x=1 and y=2, a first voltage VCAL.1 may be coupled to the node 510.1 via resistor R7.1, while a second voltage VCAL.2≠VCAL.1 may be coupled to the node 510.2 via resistor R7.2. By measuring the voltage difference between nodes IN.1 and IN.2 using amplifiers 151.1 and 151.2, an indication of the impedance between the nodes 510.1 and 510.2 may be derived.

It will be appreciated that by appropriately setting the calibration voltages VCAL.x and VCAL.y, measure the impedance between any two input nodes IN.x and IN.y may be measured. In an exemplary embodiment, the calibration voltage generation module 520 may be, e.g., a microprocessor having N separate DAC outputs that can generate programmable voltage levels. In an exemplary embodiment, the calibration voltage generation module 520 may further be provided with current measurement capability to measure the current flowing through each voltage source VCAL.1 through VCAL.N.

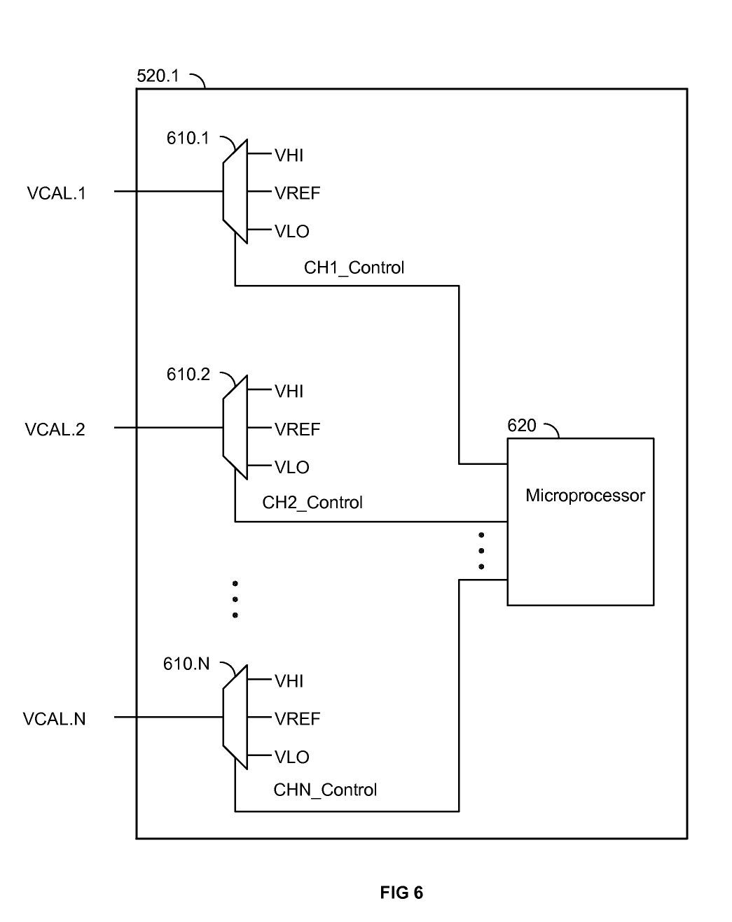

FIG. 6 illustrates an exemplary embodiment 520.1 of a calibration voltage generation module 520. Note the voltage generation module 520.1 is shown for illustrative purposes only, and is not meant to limit the scope of the present disclosure to any particular voltage generation module shown. Furthermore, it will be appreciated that certain details of the circuits described hereinabove may be omitted in FIG. 6 for simplicity.

In FIG. 6, each calibration voltage VCAL.n is generated by a corresponding multiplexer 610.n programmed to select the value of VCAL.n from amongst a plurality of inputs including Vref, a voltage VHI higher than Vref, and a voltage VLO lower than Vref. A control signal CHn_CONTROL is provided to each multiplexer 610.n to select the calibration voltage VCAL.n from amongst Vref, VHI, and VLO. In an exemplary embodiment, the impedance between the nodes IN of any two amplifier modules 151.x and 151.y, wherein x and y are integer indices from 1 to N not equal to each other, may be measured by programming the microprocessor 620 to generate suitable differential voltages VCAL.x and VCAL.y at the corresponding calibration nodes 510.n of the amplifier modules.

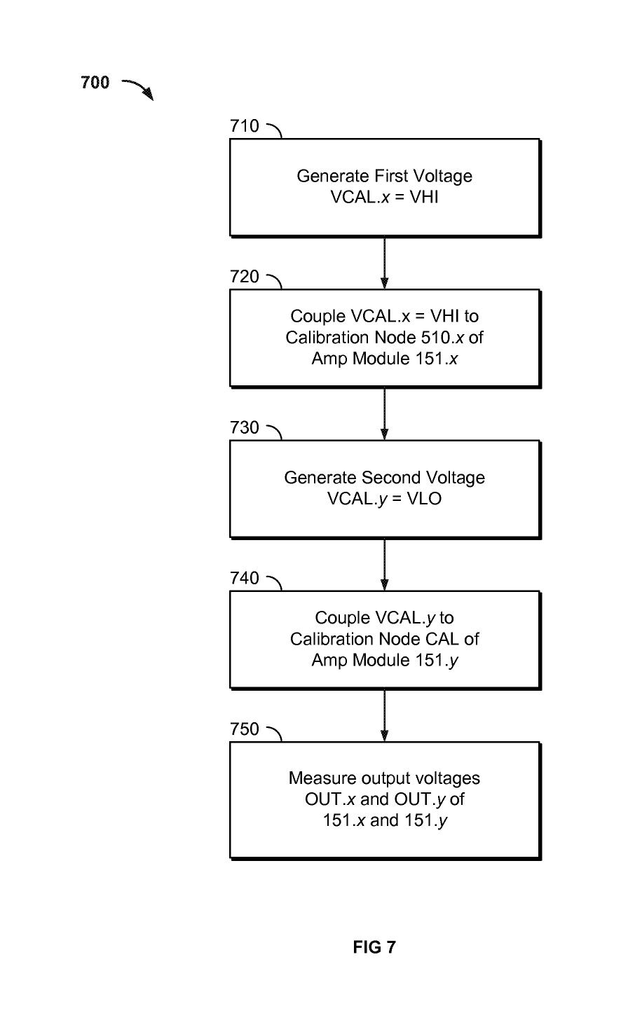

FIG. 7 illustrates an exemplary embodiment 700 of a method for measuring the input impedance between input nodes IN of two amplifier modules using the apparatus 100.2 of FIG. 5. Note the method 700 is shown for illustrative purposes only, and is not meant to limit the scope of the present disclosure to any particular method shown.

In FIG. 7, at block 710, a first voltage VCAL.x=VHI is generated by the calibration voltage generation module 520. In an exemplary embodiment, the calibration voltage generation module 520 may be implemented as embodiment 520.1 shown in FIG. 6, and the setting of VCAL.x may be performed by utilizing a multiplexer as shown in FIG. 6.

At block 720, the first voltage VCAL.x is coupled to the calibration node CAL of a first amplifier module 151.x.

At block 730, a second voltage VCAL.y=VLO is further generated by the calibration voltage generation module 520.

At block 740, the second voltage VCAL.y is coupled to the calibration node CAL of a second amplifier module 151.y.

At block 750, the output voltages OUT.x and OUT.y of amplifier modules 151.x and 151.y are measured to determine a voltage drop across IN.x and IN.y, which also provides an indication of the impedance between IN.x and IN.y.

In an exemplary embodiment, by calculating the impedance present between the IN nodes of two amplifier modules in the apparatus 100.2, i.e., the electrode contact impedance, mechanical or electrical failures resulting in, e.g., a short circuit between the inputs of two amplifier modules may be detected. Furthermore, when the apparatus 100.2 including terminals 120.1 through 120.N is implanted in a patient body, and placed in contact with, e.g., body tissue or fluid, the measured electrode contact impedance between two terminals may represent the signal source impedance of the body tissue or fluid. Data on the signal source impedance may be utilized by, e.g., the signal processing module 160, to more accurately process the voltage outputs of the amplifier modules, according to techniques derivable by one of ordinary skill in the art.

In a further aspect of the present disclosure, techniques are provided to identify mechanical and/or electrical anomalies when multiple amplifier modules are configured to amplify the difference between their corresponding inputs and the average of all amplifier module inputs. In an exemplary embodiment, each amplifier module output OUT.n is configured to be proportional (over the pass-band of the amplifier module) to the difference between the corresponding amplifier module input IN.n and the average of all amplifier module inputs IN.1 through IN.N. In this case, it is expected that the instantaneous sum of all amplifier module outputs OUT.1 through OUT.N may be equal to zero or to a reference voltage VA during normal operation. By computing the sum of the amplifier module outputs, and determining whether the sum deviates significantly from zero, anomalies in the amplifier module operation, e.g., mechanical and/or electrical anomalies, may be identified.

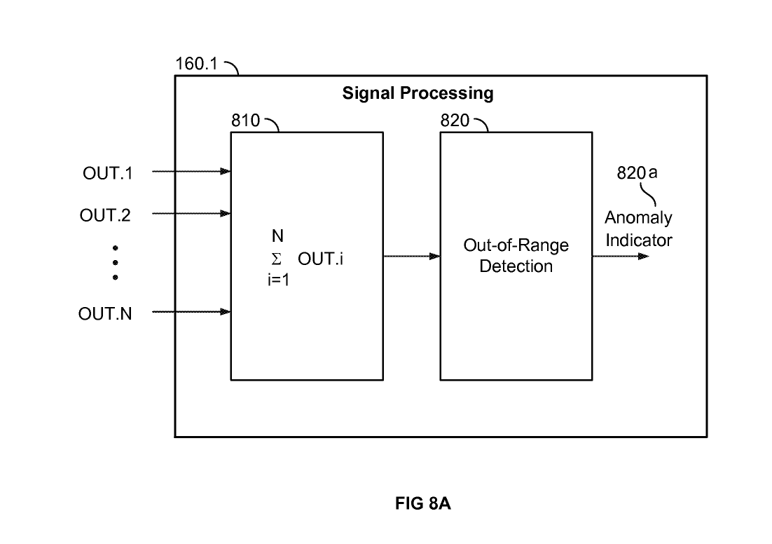

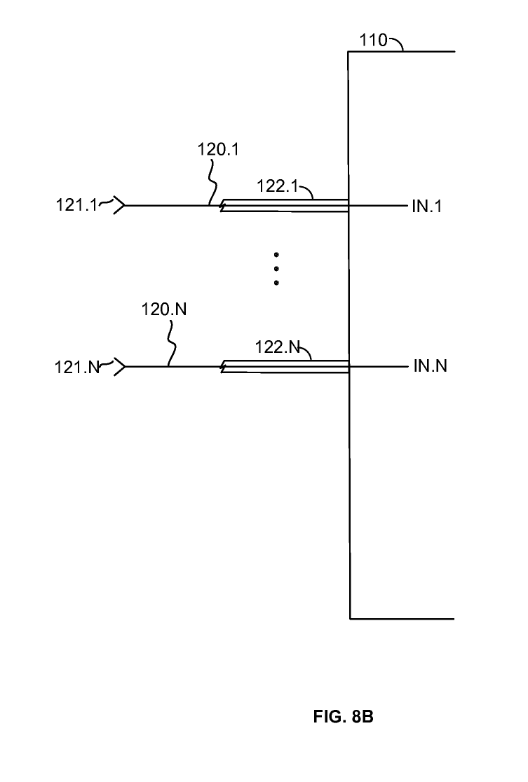

FIG. 8A illustrates an exemplary embodiment 160.1 of a signal processing module 160 configured to generate an anomaly indicator signal. In FIG. 8A, the amplifier module outputs OUT.1 through OUT.N are provided to the summation module 810, which computes the sum of all amplifier module outputs at a given time. Note the amplifier module outputs OUT.1 through OUT.N may be generated, e.g., according to the cross-coupled amplifier module configuration shown in FIG. 2. The output of the summation module 810 is provided to an out-of-range detection module 820. The module 820 may be configured to determine when the sum of the amplifier module outputs exceeds a given positive threshold, or is less than a negative threshold, and generate an anomaly indicator signal 820a indicating when the sum is out of the acceptable range. The anomaly indicator signal 820a may be used, e.g., as a diagnostic indicator to signal when a possible mechanical or electrical failure is present in the circuit. For example, the presence of an AC signal (e.g. 60 Hz) on this sum may indicate that a large common-mode signal is present.

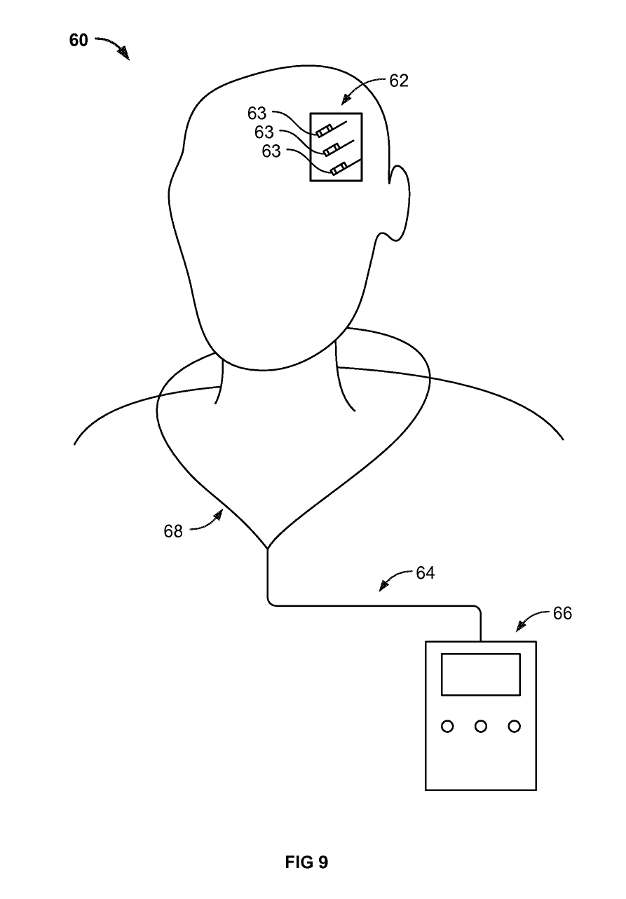

FIG. 8B illustrates an exemplary embodiment of the present disclosure, wherein insulating sheaths are provided for portions of the conducting leads external to the housing. In FIG. 9, each of the conducting leads 120.1 through 120.N is provided with a corresponding insulating sheath 122.1 through 122.N, respectively, for shielding portions of the conducting leads extending in length beyond the housing 110. The leads 120.1 through 120.N are exposed at their tips 121.1 through 121.N, respectively, to enable the lead tips to sense electrical potentials. The tips 121.1 through 121.N may include, e.g., electrodes configured to optimally contact tissue or other body surfaces. Note a base insulating sheath (not shown) may be further provided to bundle the plurality of insulating sheaths 122.1 through 122.N proximal to their origin at the housing 110.

In an exemplary embodiment, the plurality of conductive leads 120.1 through 120.N, along with corresponding insulating sheaths 122.1 through 122.N, not shown in FIG. 8B) may be bundled in a single base insulating sheath and provided as, e.g., a flexible cable having electrodes extending therefrom. The cable may have a proximal end connector (not shown) that is detachably coupleable to a corresponding connector interface (not shown) provided on the housing 110. In alternative exemplary embodiments, the single cable need not be detachably coupleable to the housing 110, and may be configured to remain fixed to the housing 110.

In exemplary embodiments, the conducting leads may be simultaneously or alternatively configured as described in co-pending U.S. Patent Publication No. 2008/0183097 A1, application Ser. No. 12/020,507, entitled “Methods and Systems for Measuring a Subject's Susceptibility to a Seizure,” filed Jan. 25, 2008, pending; U.S. patent application Ser. No. 12/630,300, entitled “Universal Electrode Array for Monitoring Brain Activity,” filed Dec. 3, 2009; and U.S. patent application Ser. No. 12/685,543, filed Jan. 11, 2010, entitled “Medical Lead Termination Sleeve for Implantable Medical Devices,” all of which are assigned to the assignee of the present disclosure, the contents of which are hereby incorporated in their entireties.

FIG. 9 depicts an exemplary embodiment of the present disclosure, wherein the techniques disclosed hereinabove are applied in the context of a real-time patient monitoring and neurological event detection system 60. Note that FIG. 9 is provided for illustrative purposes only, and is not meant to limit the application of the amplifier techniques described herein to any particular biomedical applications. For a more detailed description of the system in FIG. 9, see, e.g., “Minimally Invasive Monitoring Methods,” U.S. patent application Ser. No. 11/766,751, filed Jun. 21, 2007, U.S. Patent Publication No. 2008/0027347, pending, assigned to the assignee of the present application, the contents of which are hereby incorporated by reference in their entirety.

In FIG. 9, system 60 includes an implantable device 62 having one or more sensors or devices 63 that are configured to sample electrical activity from the patient's brain (e.g., EEG signals). The implantable device 62 may be, e.g., active (with internal power source) or semi-passive (internal power source to power components, but not to transmit data signal). The implantable device 62 may be implanted anywhere in the patient. In an exemplary embodiment, one or more of such devices may be implanted adjacent to a previously identified epileptic focus or a portion of the brain where the focus is believed to be located. Alternatively, the device 62 itself may be used to help determine the location of an epileptic focus.

In one aspect, the neural signals of the patient are sampled substantially continuously with the electrodes coupled to the electronic components of the implanted leadless device. In particular, electrical signal amplification and sampling may be performed by an apparatus such as that described hereinabove with reference to, e.g., FIGS. 1-8, with the number of electrodes corresponding to the number N of conducting leads and amplifier modules provided.

A wireless signal is transmitted that is encoded with data that is indicative of the sampled neural signal from the implanted device to an external device. The wireless signal can be any type of wireless signal, e.g., radiofrequency signal, magnetic signal, optical signal, acoustic signal, infrared signal, etc.

The physician may implant any desired number of devices in the patient. As noted above, in addition to monitoring brain signals, one or more additional implanted devices 62 may be implanted to measure other physiological signals from the patient.

The implantable device 62 may be configured to substantially continuously sample the brain activity of the groups of neurons in the immediate vicinity of the implanted device 62. The implantable device 62 may be interrogated and powered by a signal from an external device 64 to facilitate the substantially continuous sampling of the brain activity signals. Each sample of the patient's brain activity may contain between about 8 bits per sample and about 32 bits per sample, and preferably between about 12 bits per sample and about 16 bits per sample.

In alternative embodiments, it may be desirable to have the implantable devices sample the brain activity of the patient on a non-continuous basis. In such embodiments, an implantable device 62 may be configured to sample the brain activity signals periodically (e.g., once every 10 seconds) or aperiodically.

The implantable device 62 may include a separate memory module for storing the recorded brain activity signals, a unique identification code for the device, algorithms, other programming, or the like.

A patient instrumented with the implanted device 62 may carry a data collection device 64 that is external to the patient's body. The external device 64 would receive and store the signals from the implanted device 62 with the encoded EEG data (or other physiological signals). The signals received from the implanted device 62 may be represented as a multi-channel signal. The external device 64 is typically of a size so as to be portable and carried by the patient in a pocket or bag that is maintained in close proximity to the patient. In alternative embodiments, the device may be configured to be used in a hospital setting and placed alongside a patient's bed. Communication between the data collection device 64 and the implantable device 62 may take place through wireless communication. The wireless communication link between implantable device 62 and external device 64 may provide a communication link for transmitting data and/or power. External device 64 may include a control module 66 that communicates with the implanted device through an antenna 68. In the illustrated embodiment, antenna 68 is in the form of a necklace that is in communication range with the implantable devices 62.

Transmission of data and power between implantable device 62 and external device 64 may be carried out through a radiofrequency link, infrared link, magnetic induction, electromagnetic link, Bluetooth® link, Zigbee link, sonic link, optical link, other types of wireless links, or combinations thereof.

In an exemplary embodiment, the external device 64 may include software to pre-process the data according to the present disclosure and analyze the data in substantially real-time. For example, the received RF signal with the sampled EEG may be analyzed for the presence of anomalies according to the present disclosure, and further by EEG analysis algorithms to estimate the patient's brain state which is typically indicative of the patient's propensity for a neurological event. The neurological event may be a seizure, migraine headache, episode of depression, tremor, or the like. The estimation of the patient's brain state may cause generation of an output. The output may be in the form of a control signal to activate a therapeutic device (e.g., implanted in the patient, such as a vagus nerve stimulator, deep brain or cortical stimulator, implanted drug pump, etc.).

In an exemplary embodiment, the output may be used to activate a user interface on the external device to produce an output communication to the patient. For example, the external device may be used to provide a substantially continuous output or periodic output communication to the patient that indicates their brain state and/or propensity for the neurological event. Such a communication could allow the patient to manually initiate self-therapy (e.g., wave wand over implanted vagus nerve stimulator, cortical, or deep brain stimulator, take a fast acting anti-epileptic drug, etc.).

In an alternative exemplary embodiment, the external device 64 may further communicate with an auxiliary server (not shown) having more extensive computational and storage resources than can be supported in the form factor of the external device 64. In such an exemplary embodiment, anomaly pre-processing and EEG analysis algorithms may be performed by an auxiliary server, or the computations of the external device 64 may be otherwise facilitated by the computational resources of the auxiliary server.

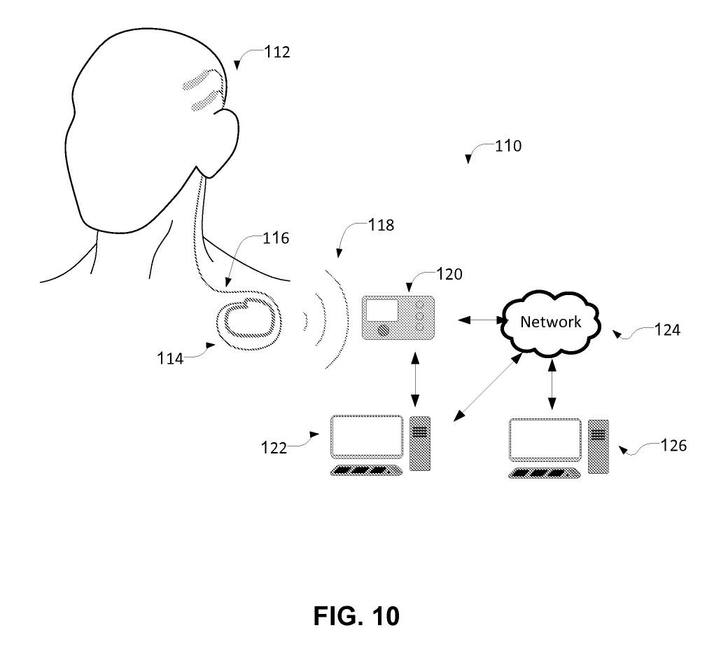

FIG. 10 depicts another exemplary embodiment, wherein the techniques disclosed hereinabove are applied in the context of a real-time patient monitoring and neurological event detection system 110. Note that FIG. 10 is provided for illustrative purposes only, and is not meant to limit the application of the techniques described herein to any particular biomedical applications. For a more detailed description of the system in FIG. 10, see, e.g., U.S. Patent Publication No. 2008/0183097 A1, application Ser. No. 12/020,507 filed Jan. 25, 2008, pending, the contents of which are hereby incorporated by reference in their entirety.

In the embodiment shown in FIG. 10, the plurality of insulated conducting leads 120.1 through 120.N may be provided as an electrode array 112. The electrode array 112 is connected to the housing of an implanted assembly 114 via leads 116. The conducting leads may be bundled in a single cable 116, and the cable 116 may be tunneled between the cranium and the scalp and subcutaneously through the neck to the implanted assembly 114. The implanted assembly 114 may be implanted in a sub-clavicular pocket in the subject, or the implanted assembly 114 may be disposed somewhere else in the subject's body. For example, the implanted assembly 114 may be implanted in the abdomen or underneath, above, or within an opening in the subject's cranium (not shown).

As shown in FIG. 10, the electrode array 112 may be positioned anywhere in, on, and/or around the subject's brain, but typically one or more of the electrodes are implanted within the subject. For example, one of more of the electrodes may be implanted adjacent or above a previously identified epileptic network, epileptic focus or a portion of the brain where the focus is believed to be located. While not shown, it may be desirable to position one or more electrodes in a contralateral position relative to the focus or in other portions of the subject's body to monitor other physiological signals.

The electrode arrays 112 may comprise one or more contacts for collecting the neurological signals. The electrode arrays 112 of the present invention may be intracranial electrodes (e.g., epidural, subdural, and/or depth electrodes), extracranial electrodes (e.g., spike or bone screw electrodes, subcutaneous electrodes, scalp electrodes, dense array electrodes), or a combination thereof. While it is preferred to monitor signals directly from the brain, it may also be desirable to monitor brain activity using sphlenoidal electrodes, foramen ovale electrodes, intravascular electrodes, peripheral nerve electrodes, cranial nerve electrodes, or the like. While the disclosure herein may focus on intracranial electrodes for sampling intracranial EEG, it should be appreciated that the present invention encompasses any type of electrodes that may be used to sample any type of physiological signal from the subject. It will be appreciated that the electrical potentials as sampled by the electrode arrays 112 may be coupled to input conductive leads and processed according to the techniques described in the present disclosure. In an aspect, the neural signals of the patient are sampled substantially continuously with the electrodes coupled to the electronic components of the implanted leadless device. In particular, electrical signal amplification and sampling may be performed by an apparatus such as that described hereinabove with reference to, e.g., FIGS. 1-9, with the number of electrodes corresponding to the number N of conducting leads and amplifier modules provided.

In the configuration illustrated in FIG. 10, two electrode arrays 112 are positioned in an epidural or subdural space, but as noted above, any type of electrode placement may be used to monitor brain activity of the subject. For example, in a minimally invasive embodiment, the electrode array 112 may be implanted between the skull and any of the layers of the scalp. Specifically, the electrodes 112 may be positioned between the skin and the connective tissue, between the connective tissue and the epicranial aponeurosis/galea aponeurotica, between the epicranial aponeurosis/galea aponeurotica and the loose aerolar tissue, between the loose aerolar tissue and the pericranium, and/or between the pericranium and the calvarium. To improve signal-to-noise ratio, such subcutaneous electrodes may be rounded to conform to the curvature of the outer surface of the cranium, and may further include a protuberance that is directed inwardly toward the cranium to improve sampling of the brain activity signals. Furthermore, if desired, the electrode may be partially or fully positioned in openings disposed in the skull. Additional details of exemplary wireless minimally invasive implantable devices and their methods of implantation can be found in U.S. Patent Publication No. 2008/0027347 A1, application Ser. No. 11/766,742, entitled “Minimally Invasive Monitoring Systems,” filed Jun. 21, 2007, pending, the disclosure of which is incorporated by reference herein in its entirety.

In an exemplary embodiment, the implanted assembly 114 may include software to pre-process the data according to the present disclosure and analyze the data in substantially real-time. For example, the sampled EEG from the electrode arrays 112 may be analyzed for the presence of anomalies according to the present disclosure, and further by EEG analysis algorithms to estimate the patient's brain state which is typically indicative of the patient's propensity for a neurological event. The neurological event may be a seizure, migraine headache, episode of depression, tremor, or the like. The estimation of the patient's brain state may cause generation of an output. The output may be in the form of a control signal to activate a therapeutic device (e.g., implanted in the patient, such as a vagus nerve stimulator, deep brain or cortical stimulator, implanted drug pump, etc.).

In an exemplary embodiment, the implanted assembly 114 may further wirelessly communicate with an external device 120 to activate a user interface and produce an output communication to the patient. For example, the external device 120 may be used to provide a substantially continuous output or periodic output communication to the patient that indicates their brain state and/or propensity for the neurological event. Such a communication could allow the patient to manually initiate self-therapy (e.g., wave wand over implanted vagus nerve stimulator, cortical, or deep brain stimulator, take a fast acting anti-epileptic drug, etc.).

In an alternative exemplary embodiment, the external device may further communicate with an auxiliary server (e.g., server 126) having more extensive computational and storage resources than can be supported in the form factor of the external device. In such an exemplary embodiment, anomaly pre-processing and EEG analysis algorithms may be performed by an auxiliary server 126, or the computations of the external device may be otherwise facilitated by the computational resources of the auxiliary server 126.

### Signal Referencing

Described above are techniques for amplifying a plurality of input voltages to generate a corresponding plurality of output voltages, in which each of the plurality of input voltages is referenced to a common voltage comprising the average of the plurality of input voltages, without the need to reference an externally provided common voltage. These techniques may be particularly useful when averaging a large number of input voltages, such as in the sixteen channel embodiments described above. If the signals on each channel are independent, the amplitude of N averaged signals is expected to be 1/(sqrt(N)) than that from a single channel. Therefore, when using sixteen independent channels, the averaged signal is expected to be 1/(sqrt(16))=¼ than that of a single channel. However, it may be desirable to use a modified version of these techniques in certain situations.

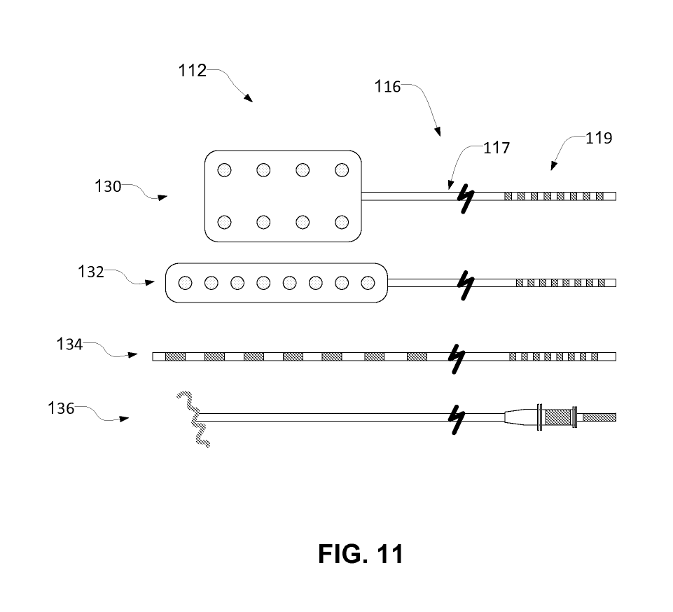

Some exemplary configurations of the electrode arrays 112 are shown in FIG. 11. Each of the illustrated electrode arrays has eight electrode contacts so as to provide sixteen channels for monitoring the EEG signals. The electrode contacts may be bipolar or referential. It should be appreciated however, that while FIG. 2 illustrates sixteen channels that are distributed over two electrode arrays, any number electrode arrays that have any number of contacts may be used. In most embodiments, however, the system typically includes between about 1 and about 256 channels, and preferably between about 1 and about 32 channels, and more preferably between 8 and 32 channels that are distributed over 1 array and about 4 arrays. The array pattern and number of contacts on each array may be configured in any desirable pattern.

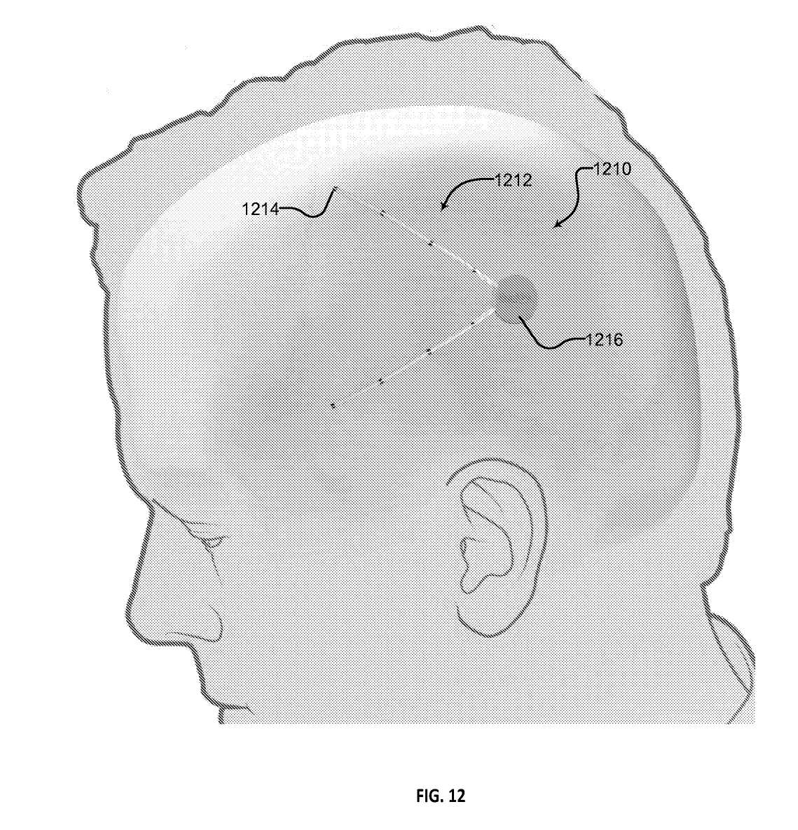

FIG. 12 illustrates a monitoring assembly 1210 that may be implanted beneath one or more layers of the scalp, but outside of the patient's skull. In the illustrated embodiment, the assembly includes two leads 1212, each lead having four electrode contacts 1214, thereby providing the assembly 1210 with the capability of monitoring eight channels of EEG data. In some embodiments, greater or fewer numbers of leads 1212, contacts 1214, and assemblies 1210 may be used.

Each lead 1212 has a proximal end coupled to an implanted collection device 1216. The implanted collection device 1216 comprises a hermetically sealed housing containing electronics for detecting and storing the physiological signals being monitored. In an exemplary embodiment, the housing may be made of titanium, ceramic, or other biocompatible material. In the illustrated embodiment, the leads 1212 are fixedly coupled to the collection device 1216. In other embodiments, the collection device 1216 includes a connector permitting the surgeon to attach or detach the leads 1212 from the collection device 1216.

The leads 1212 may take any of a variety of forms suitable for detecting the physiological signal to be monitored. Each lead 1212 is configured to come in direct physical contact with biological tissue or fluid inside the body of a patient, to sense electrical potentials present on such tissue or fluid. In the illustrated embodiment, the leads 1212 are cylindrical micro-leads approximately 1.5 mm in diameter with cylindrical contacts 14. In other embodiments, the leads 1212 may be provided in other shapes and designs, such as a strip electrode array, grid electrode array, or depth electrodes. The size and shape of each contact 1214 may also vary in different embodiments, depending on the type of signal being monitored.

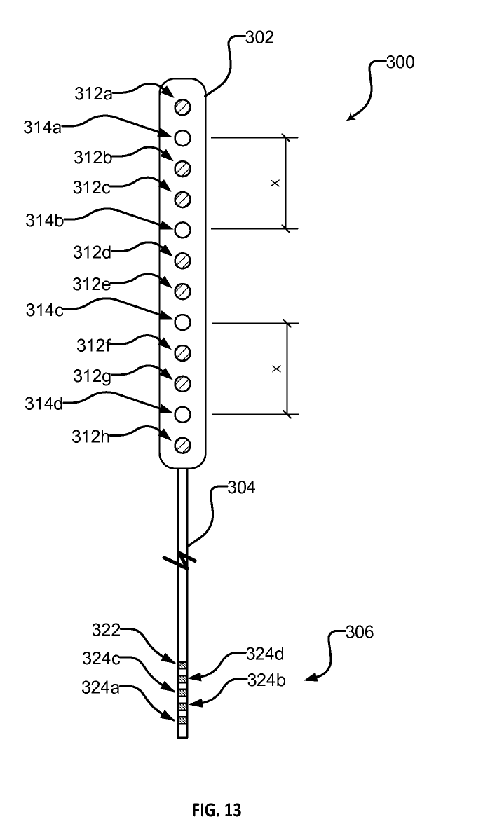

FIG. 13 illustrates a lead assembly 300 that may be used with an implantable monitoring system to carry physiological signals from the contacts to the implanted circuitry for amplifying and sampling the physiological signals. The lead assembly 300 may be used in place of the leads 1212 in monitoring assembly 1210 shown in FIG. 12, or in place of lead assembly 112 in monitoring system 110 in FIG. 10. Lead assembly 300 comprises a distal contact region 302, a lead body portion 304, and a proximal connector portion 306. The distal contact region 302 includes a plurality of contacts (e.g., contacts 312a-312h, 314a-314d) for detecting electrical signals from the patient. The proximal connector portion 306 includes one or more proximal contacts (e.g., 322, 324a-324d) for coupling with corresponding terminals in the implanted collection device. The terminals are coupled to the electronic circuitry for amplifying and sampling the detected signals. The lead body portion 304 includes an insulative cover containing one or more conductive leads for carrying electrical signals detected by the contacts in the distal contact region 302 to the proximal contacts.

In the embodiment illustrated in FIG. 13, the distal contact region 302 includes a linear array of eight reference contacts 312a-312h and four signal contacts 314a-314d, and the proximal connector portion 306 includes a single proximal reference contact 322 and four proximal signal contacts 324a-324d. Each of the four signal contacts 314a-314d are coupled to one of the four corresponding proximal signal contacts 324a-324d via one of four corresponding leads (not shown) which carry the signals through the lead body 304. This enables the lead assembly 300 to provide four channels of EEG detected by the four signal contacts 314a-314d. The signal contacts 314a-314d are separated by a distance X from the adjacent signal contact 314a-314d.

The eight reference contacts 312a-312h are coupled to a single proximal reference contact 322 via a single reference lead (not shown) which carries the signals through the lead body 304.

EEG signals are typically contaminated by various artifacts unrelated to the neurological signals of interest, for example, in a monitoring device for epilepsy patients. Electromyographic (EMG) artifacts are particularly prominent when the electrical signals are detected extracranially, such as when using externally-placed scalp electrodes, or implanted electrodes positioned outside of the skull. When designing a neurological monitoring system, it would be desirable to configure the system and its electrodes to optimize EEG detection while suppressing EMG contamination.

As a general matter, when two electrodes are positioned close together, the physiological signals detected by each electrode look similar. As the two electrodes are positioned farther apart, the differences in the physiological signals detected by each electrode increase.

When positioning electrodes extracranially, the electrode contacts are relatively far away from the EEG generators (i.e., the neurons of the brain), but close to the EMG generators (i.e., muscles in the head). Accordingly, as the distance between two extracranial electrodes increases, the EMG should decorrelate with distance faster than the EEG.

In a lead assembly comprising a linear array of N electrodes, with a spacing of X between adjacent electrodes, it may be desirable to select X and N such that the electrode array is relatively “close together” for EEG but at the same time adjacent electrode contacts are relatively “far apart” for EMG. X may be selected to be at least the minimum distance necessary to decorrelate at least a portion of the EMG signal between adjacent contacts, and N may be selected to be the number of contacts such that the EEG signal on the first contact is still reasonably well correlated with the EEG signal on the Nth contact.

The eight reference contacts 312a-312h may be summed together by connecting them to a single proximal reference contact 322. The signal from this single proximal reference contact 322 may be used alone or in combination of one or more of the other channels of data from the signal contacts 314a-314d to generate the common reference voltage for the amplification of the input signals.

The signal from the reference contact 322 may be detected in a variety of ways. For example, eight separate reference contacts 312a-312h may be connected to the same lead wire. Alternatively, a single large contact 312 may be masked so as to detect signals from multiple disparate locations. Alternatively, a single, elongate contact may be provided along the length of the contact region 302.

In embodiments described above with respect to FIGS. 1-8, each of the sixteen input voltages is referenced to a common voltage comprising the average of the sixteen input voltages, without the need to reference an externally provided common voltage. In embodiments where two lead assemblies 300 are coupled to a single monitoring device, each lead assembly 300 may contribute five channels of data—four EEG signal channels and one reference channel. Accordingly, ten input channels are provided to the EEG front end electronics—two reference channels and eight signal channels. Various combinations of these ten input channels may be used to generate the common reference voltage for amplification of the signal channels.

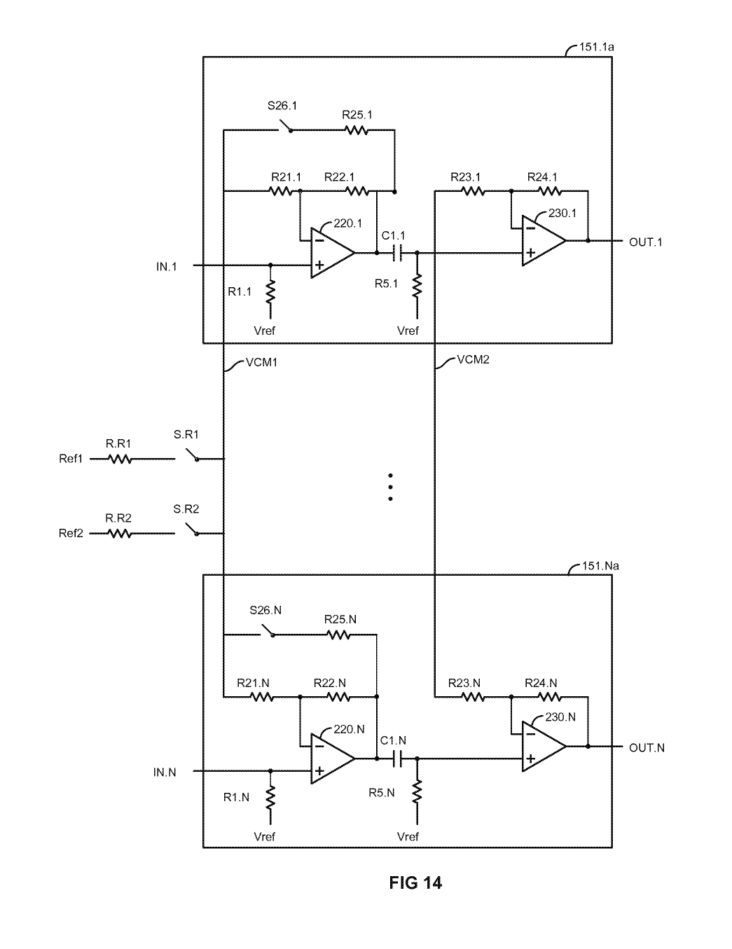

FIG. 14 illustrates a plurality of amplifier modules, similar to FIG. 2, but where the reference channels are coupled to the single common node VCM1 via switches S.R1 and S.R2. In addition, the output of each amplifier 220.1-220.N may be coupled to the common node VCM1 via resistors R25.1-R25.N and switches S26.1-S26.N (where N=8 in this example). Therefore, the signals that are used to contribute to the common node VCM2 can be selected.

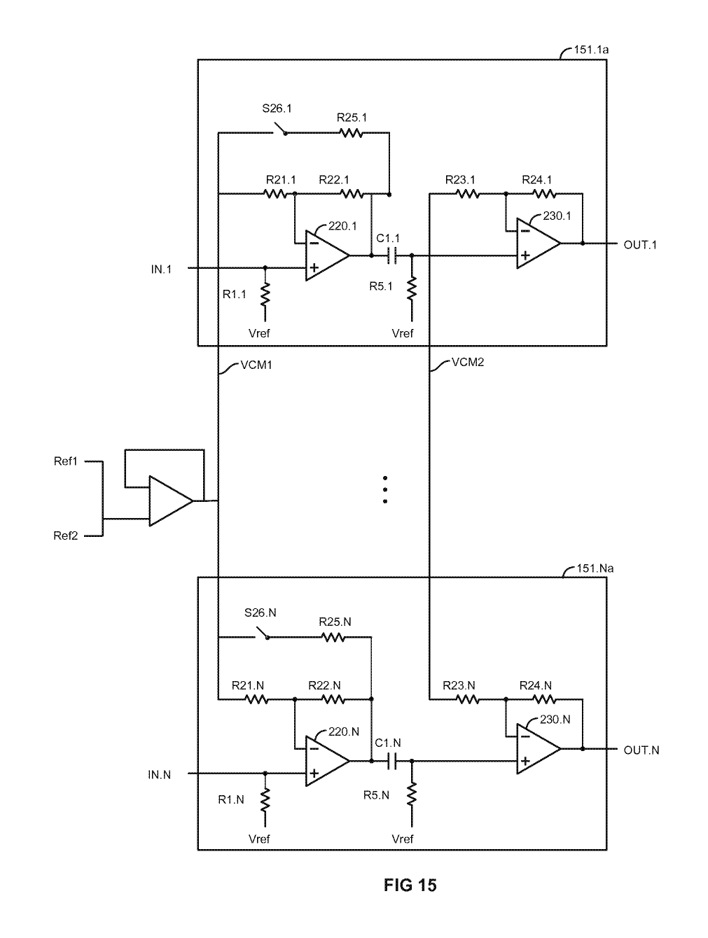

FIG. 15 illustrates a plurality of amplifier modules, similar to FIG. 14, but where the reference channels are coupled to the single common node VCM1 via an amplifier.

### Electrode Placement

In accordance with some embodiments, the positioning and orientation of an array of electrodes may be selected so as to further improving EEG detection while minimizing EMG artifact.

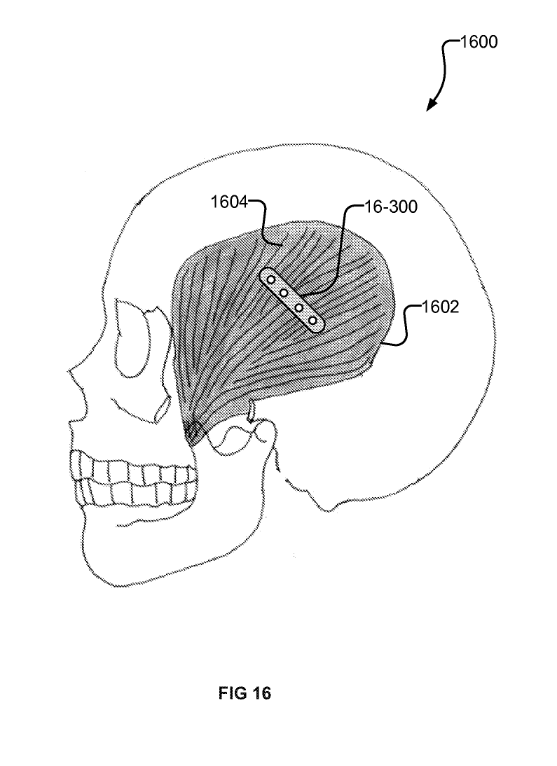

For many epilepsy patients, it is desirable to monitor EEG signals from the temporal lobe. Therefore, for a monitoring system using extracranial electrodes, the electrodes may be positioned adjacent to the temporalis muscle. FIG. 16 illustrates the placement of an electrode array 16-300 over the temporalis muscle 1602 of a patient 1600. When the patient chews or speaks, the muscle fibers 1604 of the temporalis muscle 1602 contract, thereby generating EMG artifact that may be detected by the electrode array 16-300. By positioning the electrode array 16-300 such that the individual contacts are arranged linearly orthogonal to the direction of the adjacent temporalis muscle fibers, the detected EMG may decorrelate more quickly with increasing distance between contacts than if the contacts were arranged in parallel with the fibers 1604. Such an arrangement may be used to further reduce the EMG artifact.

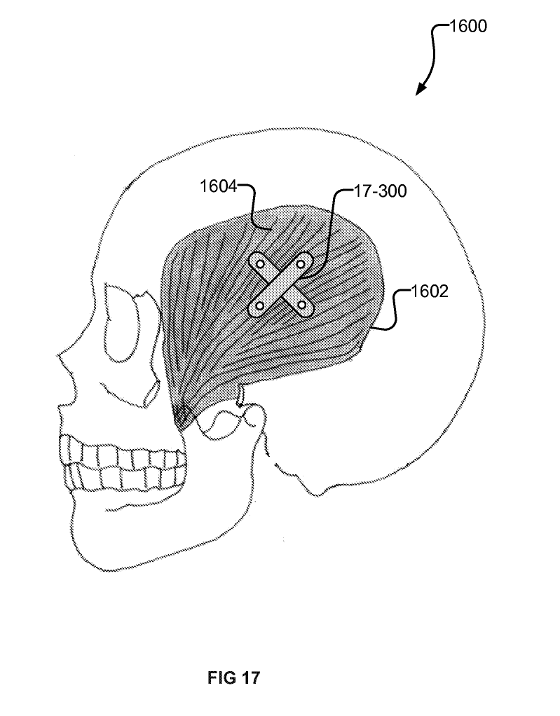

FIG. 17 illustrates the placement of an electrode array 17-300 wherein the contacts are configured in a two-dimensional pattern. In contrast with the linear array 16-300 in FIG. 16, the contacts in array 17-300 are configured to detect signals over a larger area both across and along the muscle fibers 1604. This may be desirable if precise placement of the array 17-300 relative to the muscle fibers is difficult. By analyzing the signals from the two-dimensional array of contacts, it may be possible to disassociate the signals from the muscle fibers in order to reduce EMG artifact.

Based on the teachings described herein, it should be apparent that an aspect disclosed herein may be implemented independently of any other aspects and that two or more of these aspects may be combined in various ways. In one or more exemplary embodiments, the functions described may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD/DVD-ROM or other optical disk storage, magnetic disk storage or other magnetic storage devices, solid-state flash cards or drives, or any other medium that can be used to carry or store desired program code in the form of instructions or data structures and that can be accessed by a computer. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and Blu-Ray disc where disks usually reproduce data magnetically, while discs reproduce data optically with lasers. Combinations of the above should also be included within the scope of computer-readable media.

A number of aspects and examples have been described. However, various modifications to these examples are possible, and the principles presented herein may be applied to other aspects as well.

1. A device, comprising:
a plurality of amplifier modules each having an input node and an output node, each of the plurality of amplifier modules having a first amplifier with a first amplifier output connected to a second amplifier at a second amplifier input of the second amplifier;
a first amplifier common node electrically coupling together the first amplifier outputs of each of the plurality of amplifier modules; and
a reference channel coupled to the first amplifier common node via at least one of a reference channel switch, a reference channel resistor, and a reference channel amplifier electrically disposed between the reference channel and the first amplifier common node.

2. The device of claim 1, wherein each of the plurality of amplifier modules includes at least one of a first amplifier switch and a first amplifier resistor electrically disposed between the first amplifier output and the first amplifier common node.

3. The device of claim 2, wherein at least one of the plurality of amplifier modules is configured to provide a variable output at the first amplifier output due to a status of at least one of the first amplifier switch and the first amplifier resistor.

4. The device of claim 2, wherein at least one of the plurality of amplifier modules is configured to provide a variable output at the output node due to a status of at least one of the first amplifier switch and the first amplifier resistor.

5. The device of claim 2, wherein the second amplifier includes a second amplifier output, the device further comprising:
a second amplifier common node electrically coupling together the second amplifier outputs of each of the plurality of amplifier modules.

6. The device of claim 1, further comprising:
another reference channel coupled to the first amplifier common node via at least one of another reference channel switch and another reference channel resistor electrically disposed between the another reference channel and the first amplifier common node.

7. The device of claim 1, wherein the reference channel is coupled to the first amplifier common node via the reference channel amplifier, wherein the reference channel amplifier includes a first input and a second input with the reference channel is electrically coupled to the first input, the device further comprising:
another reference channel coupled to the first amplifier common node via the first input of the reference channel amplifier.

8. The device of claim 1, wherein each of the plurality of amplifier modules includes a capacitor electrically disposed between the first amplifier output and the second amplifier input.

9. The device of claim 1, wherein the first amplifier comprises a non-inverting input, an inverting input, and the first amplifier output,
wherein the non-inverting input is coupled to the input node,
wherein the non-inverting input is coupled to the first amplifier output via a coupling impedance,
wherein the first amplifier output is coupled to the inverting input via a first feedback impedance, and
wherein the inverting input is further coupled to a first amplifier common node via an inverting input impedance.

10. A method of amplifying a signal received at each of a plurality of input nodes, each of the plurality of input nodes coupled to a corresponding output node via a first amplifier having a first amplifier output coupled to a second amplifier input of a second amplifier, the method comprising:
amplifying the signal received at the first amplifier relative to a common reference comprising an average of each of the plurality of input nodes; and
adjusting a contribution of at least one of the plurality of input nodes to the average by an operation of at least one of a first amplifier switch and a first amplifier resistor disposed between the first amplifier output and an input to the first amplifier corresponding to the at least one of the plurality of input nodes.

11. The method of claim 10, further comprising:
adjusting the common reference by modifying a coupling between at least one reference channel and the common reference, the coupling comprising at least one of a reference channel switch and a reference channel resistor.

12. The method of claim 10, further comprising:
disposing a plurality of electrodes in a linear array, each of the plurality of electrodes corresponding to one of the plurality of input nodes.

13. The method of claim 12, wherein the linear array defines an array direction along which a majority of the plurality of electrodes are disposed, the disposing of the linear array arranging the array direction orthogonal to a direction of muscle fibers disposed proximate to the array.

14. The method of claim 10, further comprising:
disposing a plurality of electrodes in a two-dimensional array, each of the plurality of electrodes corresponding to one of the plurality of input nodes.

15. The method of claim 14, wherein the two-dimension array defines first and second array directions along which a portion of the plurality of electrodes are disposed, the disposing of the two-dimensional array arranging at least one of the first and second array directions orthogonal to a direction of muscle fibers disposed proximate to the array.

16. A patient event detection system, comprising:
at least one sensor disposed to receive a signal from the patient; and
a signal amplifying device disposed to communicate with the at least one sensor, the signal amplifying device comprising:
a plurality of amplifier modules each having an input node and an output node, each of the plurality of amplifier modules having a first amplifier with a first amplifier output connected to a second amplifier at a second amplifier input of the second amplifier;
a first amplifier common node electrically coupling together the first amplifier outputs of each of the plurality of amplifier modules; and
a reference channel coupled to the first amplifier common node via at least one of a reference channel switch, a reference channel resistor, and a reference channel amplifier electrically disposed between the reference channel and the first amplifier common node.

17. The system of claim 16, wherein each of the plurality of amplifier modules includes at least one of a first amplifier switch and a first amplifier resistor electrically disposed between the first amplifier output and the first amplifier common node.

18. The system of claim 17, wherein at least one of the plurality of amplifier modules is configured to provide a variable output at the first amplifier output due to a status of at least one of the first amplifier switch and the first amplifier resistor.

19. The system of claim 17, wherein at least one of the plurality of amplifier modules is configured to provide a variable output at the output node due to a status of at least one of the first amplifier switch and the first amplifier resistor.

20. The system of claim 17, wherein the second amplifier includes a second amplifier output, the device further comprising:
a second amplifier common node electrically coupling together the second amplifier outputs of each of the plurality of amplifier modules.
